(19) United States
(12) Patent Application Publication
(10) Pub. No.: US 2025/0288264 A1
(43) Pub. Date: Sep. 18, 2025

(54) X-RAY IMAGING APPARATUS

(71) Applicant: SHIMADZU CORPORATION, Kyoto-shi (JP)

(72) Inventor: Masahiro TANAKA, Kyoto-shi (JP)

(21) Appl. No.: 19/067,166

(22) Filed: Feb. 28, 2025

(30) Foreign Application Priority Data
Mar. 13, 2024 (JP) 2024-038868

(51) Int. Cl. A61B 6/04; A61B 6/10

(52) U.S. Cl. CPC A61B 6/0487; A61B 6/10

(57) ABSTRACT

[Problem] Provided is an X-ray imaging apparatus that can reduce a burden on an operator and time required for examination.

[Solution] Provided are an X-ray tube 5 that irradiates a subject M with X-rays, an X-ray detector 7 that detects X-rays transmitted through the subject M, an operating unit 19 that is located at a position where a level thereof changes together with a level of at least either the X-ray tube 5 or the X-ray detector 7 constituting an imaging system and receives input operation by an operator S, an optical camera 21 that acquires information on a physique of the operator S, and a level control unit 45 that controls a level of the operating unit 19 to be a level F1 suitable for the input operation by the operator S in accordance with the information on the physique.

### TECHNICAL FIELD

The present invention relates to an X-ray imaging apparatus.

As for a currently-used X-ray imaging apparatus, a liftable imaging apparatus is known that includes a table on which a subject is placed, a base that supports the table, and a lifting drive unit that moves the base upward and downward. In such an imaging apparatus, a body of the apparatus includes a console that receives input operation concerning driving of the table or an imaging system. The console is arranged on a front face of the table or the base, for example, and mainly accepts input operations for adjusting a range of X-rays (e.g., Patent Literature 1).

When performing a procedure like a puncture or endoscopy using the X-ray imaging apparatus as one example, an operator needs to perform various actions in the vicinity of the subject. In other words, the operator performs a procedure like a puncture on the subject that is placed on the table while adjusting the imaging area of X-rays with the console.

### PRIOR ART DOCUMENT

### Patent Literature

### [Patent Literature 1]

Japanese Unexamined Patent Publication No. 2021-023545

### SUMMARY OF INVENTION

### Technical Problem

However, the conventional example with such a configuration as above possesses the following drawbacks.

In the currently-used X-ray imaging apparatus, the console moves with the table, the base, or the like. Accordingly, it is feared that the console will move to a position where it is difficult for the operator to operate as a result of operating the body of the apparatus, leading to an increased burden on the operator. Specific examples of such a burden include a process of operating the console to adjust the table to a proper level after placing the subject on the table. In this case, in order to place the subject on the table easily, the console is moved to a lower position with the table at the time of placing the subject on the table. Consequently, in order to adjust the table to the proper level, the operator needs to make a motion of bending at the waist or extending his/her arm largely toward the console at the lower position. Such a motion is burdensome for the operator, and also causes a prolonged procedure.

Another configuration of the currently-used X-ray imaging apparatus for adjusting the level of the console arranged in the body of the apparatus is exemplified by moving the console upward and downward through remote control by a technologist different from the operator. In the remote control, the operator can avoid a burdensome motion. However, in the configuration of performing the remote control, the operator needs to move the console upward and downward to a level suitable for the operator by visually observing the operator and the console from away while communicating with the operator. In this approach, it is difficult for the operator to accurately understand the level suitable for the operator. As a result, a motion to adjust the level of the console finely is repeated more frequently, which may increase a burden on the operator and the operator. Extended time required for examination is also feared.

The present invention has been made regarding the state of the art noted above, and its object is to provide an X-ray imaging apparatus that can reduce a burden on an operator and time required for examination.

### Solution to Problems

The present invention is constituted as stated below to achieve the above object.

One aspect of the present invention provides an X-ray imaging apparatus, including an X-ray tube that irradiates a subject with X-rays, an X-ray detector that detects X-rays transmitted through the subject, an operating unit that is located at a position where a level thereof changes together with a level of at least either the X-ray tube or the X-ray detector constituting an imaging system and accepts input operation by an operator, a physique information acquiring unit that acquires information on a physique of the operator, and a level control unit that controls the level of the operating unit to be suitable for the input operation by the operator in accordance with the information on the physique.

### Advantageous Effects of Invention

The X-ray imaging apparatus according to the aspect of the present invention includes the operating unit that accepts input operation by the operator, the physique information acquiring unit that acquires the information on the physique of the operator, and the level control unit. The level control unit controls the level of the operating unit to be suitable for input operation by the operator in accordance with the information on the physique of the operator that the physique information acquiring unit acquires.

That is, when the physique information acquiring unit acquires the information on the physique of the operator, the level of the operating unit is controlled automatically to be suitable for input operation by the operator. Accordingly, even if a console is moved to a position where it is difficult for the operator to operate due to a motion such as placing the subject on the X-ray imaging apparatus, the operating unit is automatically moved to a suitable level by the physique information acquiring unit and the level control unit. This can avoid an increased burden on the operator due to movement of the operating unit to a position where it is difficult for the operator to operate. This can also prevent extended time required for a procedure on the subject.

### BRIEF DESCRIPTION OF DRAWINGS

FIG. 1 is a front view illustrating an overall configuration of an X-ray imaging apparatus according to a first embodiment.

FIG. 2 is a right side view illustrating the configuration of the X-ray imaging apparatus according to the first embodiment.

FIG. 3 is a functional block diagram illustrating the configuration of the X-ray imaging apparatus according to the first embodiment.

FIG. 4 is a flow chart illustrating an operation of the X-ray imaging apparatus according to the first embodiment.

FIG. 5 is a front view illustrating a process of a step S1 according to the first embodiment.

FIG. 6 is a right side view illustrating a process of a step S2 according to the first embodiment.

FIG. 7 is a right side view illustrating a process of a step S3 according to the first embodiment.

FIG. 8 is a right side view illustrating the process of the step S3 according to the first embodiment.

FIG. 9 is a right side view illustrating a process of a step S5 according to the first embodiment.

FIG. 10 is a right side view illustrating the process of the step S5 according to the first embodiment.

FIG. 11 is a right side view illustrating a process of a step S6 according to the first embodiment.

FIG. 12 is a front view illustrating an overall configuration of an X-ray imaging apparatus according to a second embodiment.

FIG. 13 is a functional block diagram illustrating the configuration of the X-ray imaging apparatus according to the second embodiment.

FIG. 14 is a flow chart illustrating an operation of the X-ray imaging apparatus according to the second embodiment.

FIG. 15 is a right side view illustrating a process of a step S104 according to the second embodiment.

FIG. 16 is a right side view illustrating a process of a step S106 according to the second embodiment.

FIG. 17 is a right side view illustrating a process of a step S107 according to the second embodiment.

FIG. 18 is a front view of a state where an angle of the table is changed in the first embodiment.

FIG. 19 is a front view of a state where a level of an operating unit is controlled in the first embodiment.

FIG. 20 is a front view illustrating a configuration of a physique information acquiring unit according to one modification.

FIG. 21 is a plan view illustrating a configuration of an input device according to another modification.

### DESCRIPTION OF EMBODIMENTS

### First Embodiment

The following describes an X-ray imaging apparatus 1 according to a first embodiment of the present invention with reference to drawings.

### <Description of Overall Construction>

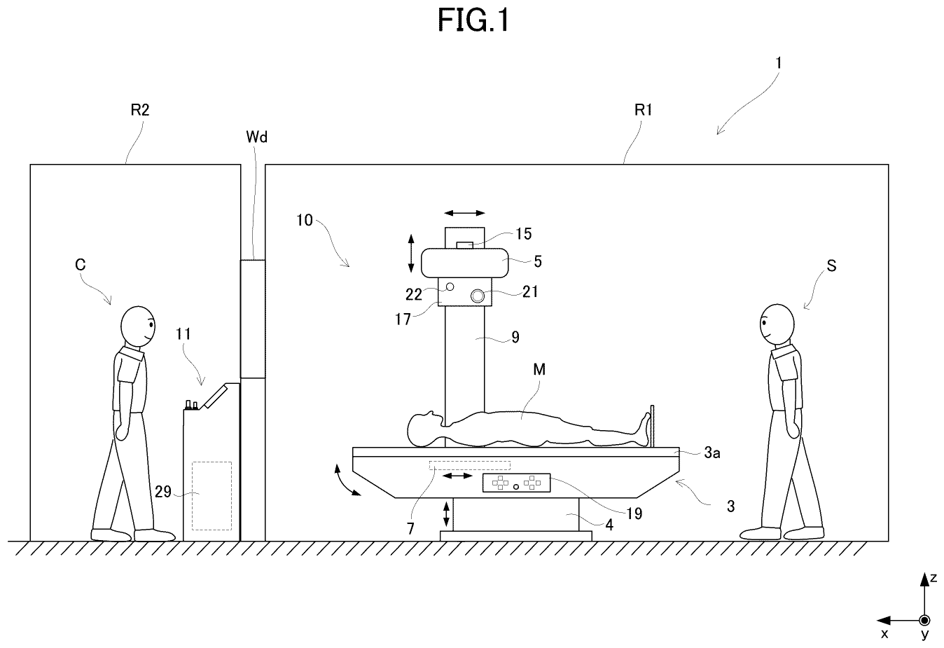

As shown in FIG. 1, the X-ray imaging apparatus 1 according to the first embodiment includes an apparatus body 10 and an input device 11. The apparatus body 10 is located in a radiography room R1, and the input device 11 is located in a control room R2. An operator S, who is for example a physician, enters the radiography room R1 with a subject M as an object of radiography, and performs various tasks. A technologist C, who is for example a radiologic technologist, enters the control room R2, and operates the input device 11.

The apparatus body 10 includes a table 3, an X-ray tube 5, an X-ray detector 7, and a strut 9. On the table 3, the subject M as an object of radiography is placed. The subject M is placed on a placement surface 3a located at an upper end of the table 3. The table 3 is supported by a base 4 arranged on the floor of the radiography room R1, and is configured to be rotatable around an axis in a shorter direction of the table 3. The base 4 is configured to be movable upward and downward, and a level of the table 3 is adjusted by up-down movement of the base 4. Now, FIG. 1 shows the table 3 in a horizontal state. In FIG. 1 and the like, it is assumed that a longer direction of the table 3 is an x-direction and the shorter direction of the table 3 is a y-direction. It is also assumed that an orthogonal direction with respect to the placement surface 3a of the table 3 is a z-direction.

The X-ray tube 5 is located above the table 3. The X-ray tube 5 irradiates the subject M with X-rays. The table 3 has an X-ray detector 7 embedded therein so as to face the X-ray tube 5. That is, when the table 3 moves upward and downward, the X-ray detector 7 moves upward and downward integrally with the table 3. The X-ray detector 7 detects X-rays emitted from the X-ray tube 5, and outputs an X-ray detection signal. The X-ray detector 7 faces the X-ray tube 5 across the table 3. Examples of the X-ray detector 7 include a flat panel detector and an image intensifier.

The strut 9 extends in a direction that intersects the table 3. A lower end of the strut 9 is connected to the table 3 via a connecting portion 13. The strut 9 is guided by a guide rail, not shown, arranged on the table 3, and is configured to be movable in the longer direction of the table 3. Here, the strut 9 may be connected to the table 3 directly or connected indirectly by a member different from the connecting portion 13.

A first end of a branch portion 15 extending in the shorter direction of the table 3 is attached to an upper end of the strut 9. The branch portion 15 is configured to be movable upward and downward along the strut 9, and the X-ray tube 5 is connected to a second end of the branch portion 15. The X-ray tube 5 is guided by a guide, not shown, arranged on the branch portion 15, and is movable in the shorter direction of the table 3. In other words, the X-ray tube 5 is movable in each of the x-direction, the y-direction, and the z-direction in response to movement of the strut 9 and the branch portion 15.

The connecting portion 13 is connected to the strut 9 and also to the X-ray detector 7. The connecting portion 13 is guided by a guide rail embedded in the table 3 and the strut 9, and is movable in the shorter direction of the table 3 synchronously with the X-ray tube 5.

That is, the imaging system consisted by the X-ray tube 5 and the X-ray detector 7 is connected to the table 3 via the strut 9, the connecting portion 13, and the branch portion 15, and is configured to be movable synchronously in the x-direction and the y-direction individually.

A collimator 17 is provided at the bottom of the X-ray tube 5. The collimator 17 limits X-rays emitted from the X-ray tube 5 to a predetermined shape. Examples of the predetermined shape include a cone in a pyramid.

The input device 11 is used for inputting instructions of the technologist C. Examples of the input device 11 include a keyboard input type panel, a touch input type panel, a pushbutton switch, and a changeover switch. Examples of information inputted by the input device 11 include a value of a source to image distance (SID), which is a distance from the X-ray tube 5 to the X-ray detector 7, a tube voltage and tube current when the X-ray tube 5 emits X-rays, and an irradiation start time. The technologist C inputs various instructions to the X-ray imaging apparatus 1 by operating the input device 11 as appropriate while checking the subject M and the apparatus body 10 through a window Wd from the control room R2.

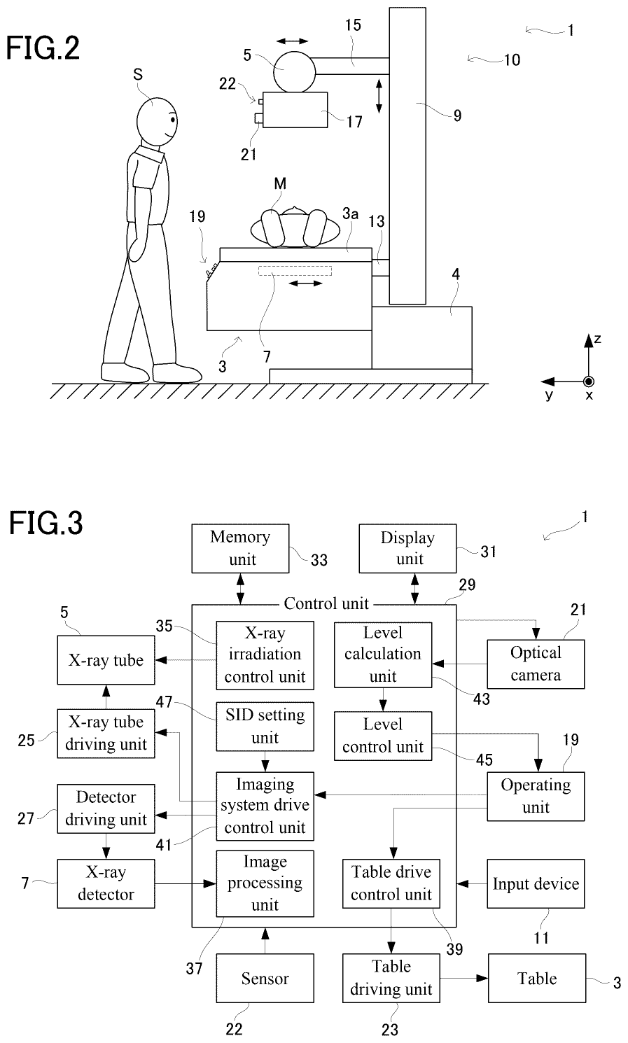

As shown in FIGS. 1 to 3, the X-ray imaging apparatus 1 includes an operating unit 19, an optical camera 21, a sensor 22, a table driving unit 23, an X-ray tube driving unit 25, a detector driving unit 27, a control unit 29, a display unit 31, and a memory unit 33.

The operating unit 19 is configured to accept input operation of the operator S concerning driving of the table 3 and the imaging system. That is, the operating unit 19 accepts the input operation, whereby a position and an angle of the table 3 and a position and an angle of the imaging system are adjusted. By changing the position and the angle of the table 3 or the imaging system as appropriate, an area where X-rays are emitted from the X-ray tube 5 can be adjusted. That is, the operator S in the radiography room R1 operates the operating unit 19 as appropriate, thereby adjusting the area where X-rays are emitted from the X-ray tube 5. Examples of the operating unit 19 include a touch input type panel, a pushbutton switch, and a changeover switch.

The operating unit 19 is located at a position whose level changes together with a level of at least one constituting the imaging system. In the first embodiment, the operating unit 19 is located on a front face of the table 3. The table 3 has the X-ray detector 7 embedded therein, and a level of the X-ray detector 7 changes together with the level of the table 3. That is, in the first embodiment, the level of the operating unit 19 changes together with the level of the X-ray detector 7.

The optical camera 21 captures optical images of the operator S. Examples of the optical camera 21 include a digital camera with a complementary metal oxide semiconductor (CMOS) image sensor built therein. A sensor 22 detects that the operator S is moving to an imaging area of the optical camera 21. The X-ray imaging apparatus 1 according to the first embodiment is configured to capture an optical image of the operator S by detecting the operator S with the sensor 22, whereby operating the optical camera 21. Examples of the sensor 22 include an infrared sensor and a temperature sensor.

In the first embodiment, the optical camera 21 and the sensor 22 are located on a front face of the collimator 17. That is, the X-ray imaging apparatus 1 according to the first embodiment is configured to capture an optical image of the operator with the optical camera 21 by approach of the operator S to a front face of the apparatus body 10. The optical image of the operator S is used for obtaining information on an optimum value F1 for the level of the operating unit 19. Here, the optimum value F1 corresponds to the level of the operating unit 19 that is suitable for input operation by the operator S with use of the operating unit 19.

The table driving unit 23, the X-ray tube driving unit 25, and the detector driving unit 27 each include a drive component such as a motor. The table driving unit 23 is provided in the base 4 as one example. The table driving unit 23 moves the table 3 in the x-direction, the y-direction, and the z-direction individually. Moreover, the table driving unit 23 turns the table 3 around an axis in the y-direction, thereby changing the angle of the table 3 with respect to a horizontal plane.

The X-ray tube driving unit 25 is provided in the strut 9 and the branch portion 15 as one example. The X-ray tube driving unit 25 drives the X-ray tube 5 in the x-direction, the y-direction, and the z-direction individually. Moreover, the X-ray tube driving unit 25 turns the X-ray tube 5 around an axis in the x-direction and an axis in the y-direction. The X-ray tube 5 turns, whereby an irradiation angle of X-rays with respect to the placement surface 3a is changed. The detector driving unit 27 is provided inside of the table 3 or at the connecting portion 13 as one example. The detector driving unit 27 drives the X-ray detector 7 in the x-direction, the y-direction, and the z-direction individually.

The control unit 29 includes an information processing device such as a central processing unit (CPU) as one example. The control unit 29 performs overall control of operation of various components that constitute the X-ray imaging apparatus 1 in accordance with input operation accepted by the input device 11 or the operating unit 19. As shown in FIG. 1, the control unit 29 is embedded in the input device 11 as one example. A position of the control unit 29 in the X-ray imaging apparatus 1 is not limited to the input device 11, but may be changed as appropriate.

The control unit 29 includes an X-ray irradiation control unit 35, an image processing unit 37, a table drive control unit 39, an imaging system drive control unit 41, a level calculation unit 43, a level control unit 45, and an SID setting unit 47.

An X-ray irradiation control unit 35 controls various types of operation of emitting X-rays from the X-ray tube 5. That is, the X-ray irradiation control unit 35 controls a dose of X-rays emitted from the X-ray tube 5 and a timing of emitting X-rays by controlling the tube voltage, the tube current, and the like applied to the X-ray tube 5.

An image processing unit 37 performs various types of image processing based on the X-ray detection signal outputted from the X-ray detector 7 to generate an X-ray image. The table drive control unit 39 controls operation of the table driving unit 23. An imaging system drive control unit 41 controls operation of the X-ray tube driving unit 25 and the detector driving unit 27 overall.

The level calculation unit 43 uses the optical image of the operator S taken by the optical camera 21 to calculate an optimum value F1 for the level of the operating unit 19. As one example of how the level calculation unit 43 calculates the optimum value F1, a method is included where the level calculation unit 43 identifies physique information on the operator S from the optical image of the operator S. Examples of the physique information on the operator S include a body height of the operator S, a shoulder height of the operator S, and an arm length of the operator S.

In general, the larger the physique of the operator S becomes, the higher the level of the operating unit 19 corresponding to the optimum value F1 is. Therefore, by obtaining the physique information of the operator S, the optimum level of the operating unit 19 can be calculated as the optimum value F1 suitable for the process that the operator S reaches for the operating unit 19 and operates the operating unit 19. As a specific example, the level calculation unit 43 can calculate the optimum value F1 by multiplying a predetermined coefficient a1 by a value h1 of the body height of the operator S.

The level control unit 45 receives the information on the optimum value F1 calculated by the level calculation unit 43. Then, the level control unit 45 controls a level of the operating unit 19 to be the optimum value F1. In the first embodiment, the operating unit 19 is arranged on the front face of the table 3, and the level thereof is changed together with the level of the table 3. Consequently, the level control unit 45 according to the first embodiment controls the table drive control unit 39, thereby adjusting the level of the operating unit 19 to the optimum value F1.

An SID setting unit 47 sets a value of the SID suitable for imaging the subject M based on the information that the technologist C enters with the input device 11. In the following description, the value of the SID set by the SID setting unit 47 is referred to as a “set SID value d1”.

The set SID value d1 that is set by the SID setting unit 47 is configured to be transmitted to the imaging system drive control unit 41. The imaging system drive control unit 41 performs control of the X-ray tube driving unit 25 and the detector driving unit 27 such that a distance between the X-ray tube 5 and the X-ray detector 7 is constantly the set SID value d1. That is, when the level control unit 45 changes the level of the operating unit 19, the imaging system drive control unit 41 controls the level of the X-ray tube 5 or the X-ray detector 7 to keep the set SID value d1.

The display unit 31 displays various types of information on the X-ray imaging apparatus 1, various types of parameter information on radiography, or X-ray images generated by the image processing unit 37. Examples of the display unit 31 include a liquid crystal monitor and a high-grade display.

The memory unit 33 writes and stores information on imaging of the subject M, various X-ray images generated by the image processing unit 37, information on the optimum value F1 obtained by the optical camera 21 and the level calculation unit 43, or various types of information on operation of the X-ray imaging apparatus 1. The stored information is read out as needed, and is outputted via a control unit 29 to the display unit 31 and the like. The memory unit 33 is constituted by a storage medium such as a nonvolatile memory as an example.

### <Description of Operation>

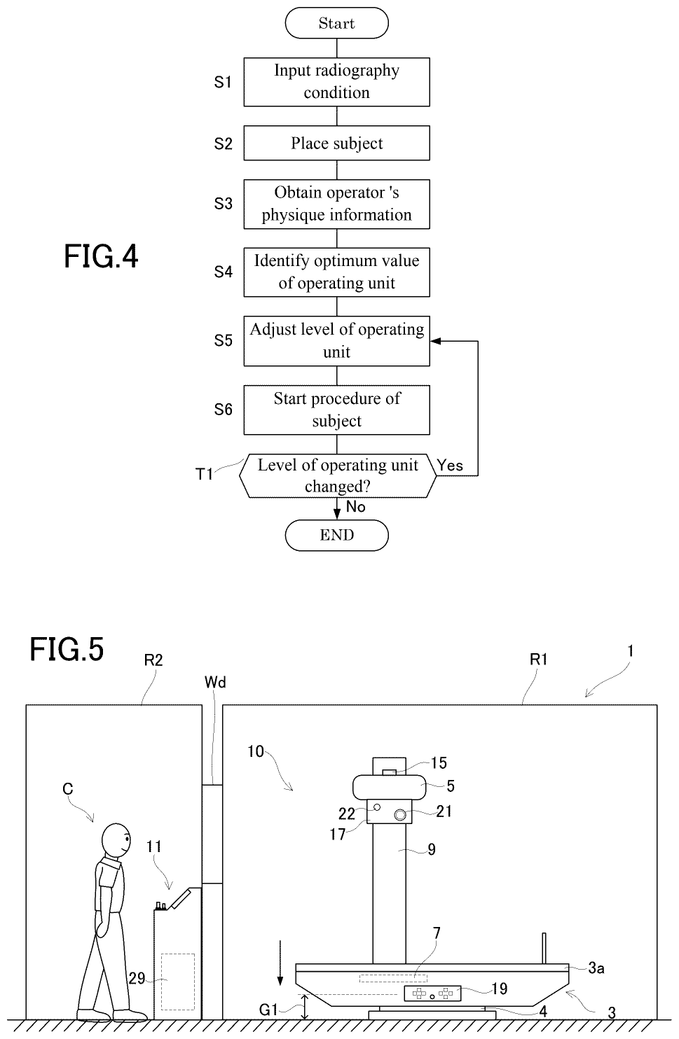

Next, description will be made of basic operation of the X-ray imaging apparatus 1 according to the first embodiment. FIG. 4 is a flow chart illustrating a summary of operation of a fluoroscopic X-ray apparatus 1 according to the first embodiment. As an example of a procedure in the first embodiment, description will be made of a case where endoscopy is performed to the subject M while an X-ray image is taken.

### Step S1 (Input Radiography Condition)

When the X-ray imaging apparatus 1 starts operation, the technologist C firstly inputs a radiography condition. When the step S1 starts, the technologist C enters the control room R2, and activates the input device 11. Then, the technologist C operates the input device 11 to input the radiography condition of the subject M while referring to an electronic medical record and the like of the subject M. Examples of the imaging condition inputted by the technologist C include information on values of the tube voltage and the tube current, an SID value, and a type of imaging.

The information on the SID that is inputted by the technologist C with the input device 11 is transmitted to the SID setting unit 47 in the control unit 29. The SID setting unit 47 sets the received value of the SID as the set SID value d1. The information on the set SID value d1 is transmitted from the SID setting unit 47 to the imaging system drive control unit 41. The imaging system drive control unit 41 controls the X-ray tube driving unit 25 to adjust the level of the X-ray tube 5 such that the distance between the X-ray tube 5 and the X-ray detector 7 becomes the set SID value d1. That is, the branch portion 15 moves upward and downward along the strut 9 under control of the imaging system drive control unit 41. The branch portion 15 moves upward and downward, whereby the level of the X-ray tube 5 is adjusted.

Moreover, the technologist C operates the input device 11 to move the table 3 downward in advance before the subject M enters the radiography room R1. An instruction inputted by the technologist C with the input device 11 is transmitted to the table drive control unit 39. The table drive control unit 39 controls the table driving unit 23, whereby the table 3 moves downward as shown in FIG. 5. By moving the table 3 downward in advance, activity of placing the subject M on the table 3 can be performed smoothly.

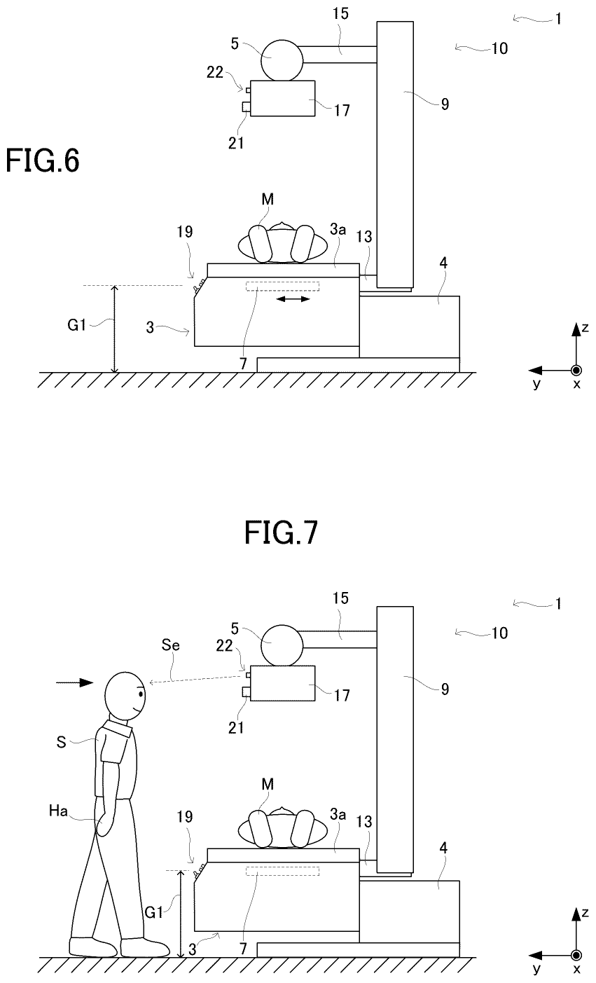

The table 3 has the operating unit 19 and the X-ray detector 7 arranged thereon. Consequently, the table 3 moves downward, whereby the level of the X-ray detector 7 and also the level of the operating unit 19 are decreased. That is, moving the table 3 downward in the step S1 lowers the operating unit 19 to a level G1, as shown in FIGS. 5 and 6.

Step S2 (Place subject)

When the technologist C inputs a radiography condition, the operator S enters the radiography room R1 with the subject M. Then, the operator S places the subject M on the placement surface 3a of the table 3. FIG. 6 shows a state where the subject M is placed on the table 3 moved downward to a lower position in advance.

In the step S2, the level of the operating unit 19 is lowered to the G1. When the table 3 is lowered and the operating unit 19 is at the level G1, it is suitable for operation of placing the subject M on the table 3 whereas it is not suitable for operation of the operating unit 19 by the operator S. That is, when the operating unit 19 is lowered to the level G1, a hand Ha of the operator S in a standing posture cannot reach the operating unit 19 (see FIG. 7). Accordingly, it is difficult for the operator S to operate the operating unit 19 in the standing posture.

When the operator S tries to operate the operating unit 19 at the level G1, the operator S needs to bend at the waist toward the operating unit 19 at the lower position, for example. Such a posture is burdensome for the operator S.

It is possible for the technologist C to move the table 3 upward by operating the input device 11 to raise the operating unit 19 from the level G1. However, in this case, it is difficult for the technologist C who is different from the operator S to determine the level suitable for the operator S to operate the operating unit 19. As a result, operation of adjusting the level of the operating unit 19 by operating the input device 11 is repeated while the operator S and the technologist C repeatedly communicate with each other. Such a communication is bothersome for both the technologist C and the operator S, and also leads to prolonged time required for the procedure.

Here in the X-ray imaging apparatus 1 according to the first embodiment, the level of the operating unit 19 is automatically controlled to an appropriate level for the operator S by the process of the step S3 to the step S5.

### Step S3 (Obtain Operator's Physique Information)

When the subject M is placed on the table 3, the process of the step S3 starts. That is, as shown in FIG. 7, the operator S moves to a front side of the table 3 to perform a procedure on the subject M. The operator S moves to the front side of the table 3 and approaches the subject M, thereby moving into an imaging area of the optical camera 21.

The sensor 22 constantly detects the front side of the table 3. When the operator S moves to the front side of the table 3, the operator S is positioned within a detection area of the sensor 22. As shown by a numeral Se in FIG. 7, the sensor 22 detects that the operator S moves into the imaging area of the optical camera 21.

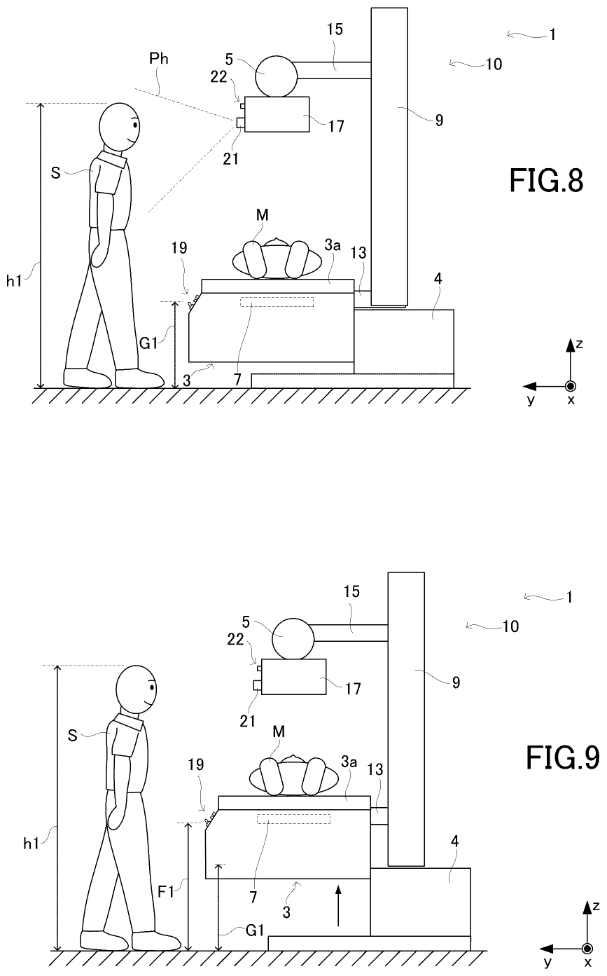

Information detected by the sensor 22 is transmitted from the sensor 22 to the control unit 29. The control unit 29 receives information that the operator S is present within the imaging area of the optical camera 21, thereby controlling the optical camera 21. As shown by a numeral Ph in FIG. 8, the optical camera 21 receives a control signal from the control unit 29, and captures an optical image of the operator S. The optical image of the operator S shows an image of the operator S. That is, the optical image of the operator S includes physique information of the operator S. Consequently, by capturing the optical image of the operator S, the X-ray imaging apparatus 1 can obtain information on the physique of the operator S.

### Step S4 (Identify Optimum Value of Operating Unit)

When the optical camera 21 captures the optical image of the operator S, a process of the step S4 starts. That is, data of the optical image of the operator S is transmitted from the optical camera 21 to the level calculation unit 43.

The level calculation unit 43 analyzes the optical image of the operator S, and calculates an optimum value F1. In the first embodiment, by analyzing the optical image of the operator S, the level calculation unit 43 identifies a body height h1 of the operator S.

In general, the larger the physique of the operator S becomes, the higher the level of the operating unit 19 corresponding to the optimum value F1 is. Accordingly, the level calculation unit 43 calculates the optimum value F1 for the level of the operating unit 19 by multiplying the predetermined coefficient a1 by the value h1 of the body height of the operator S. That is, by using the optical image of the operator S, the optimum value F1, which is the level of the operating unit 19 suitable for the process of operating the operating unit 19 by the operator S, is identified.

### Step S5 (Adjust Level of Operating Unit)

When the optimum value F1 of the operating unit 19 is identified by the level calculation unit 43, the process of the step S5 starts. That is, information on the optimum value F1 for the level of the operating unit 19 is transmitted from the level calculation unit 43 to the level control unit 45.

The level control unit 45 controls operation of the various components of the apparatus body 10 so that the level of the operating unit 19 becomes the optimum value F1. In the first embodiment, the operating unit 19 is arranged on the table 3. Accordingly, the level control unit 45 transmits a control signal to the table drive control unit 39. The table drive control unit 39 controls the table driving unit 23 to move the table 3 upward. As a result, as shown in FIG. 9, the operating unit 19 moves upward with the table 3, and the level of the operating unit 19 goes from the level G1 to the optimum value F1.

By performing the process of the steps S3 to S5 in such a manner, the level of the operating unit 19 is automatically adjusted to the optimum value F1 when the operator S moves into the imaging area of the optical camera 21. In other words, the level of the operating unit 19 is automatically adjusted to the optimum value F1 while the operator S keeps his/her standing posture. Thus, the level of the operating unit 19 can be adjusted to a position suitable for a physique of the operator S without troublesome movement such as bending at the waist of the operator S.

Now, the table 3 moves upward with the operating unit 19, whereby the X-ray detector 7 arranged on the table 3 also moves upward. As a result, the X-ray detector 7 is closer to the X-ray tube 5, resulting in a decreased SID value. Consequently, in the first embodiment, the process of keeping the distance between the X-ray detector 7 and the X-ray tube 5 at the set SID value d1 is performed along with the operation of adjusting the level of the operating unit 19. That is, when the level of the operating unit 19 increases from the level G1 to the optimum value F1, the SID setting unit 47 transmits a control signal to the imaging system drive control unit 41 to keep the distance between the X-ray detector 7 and the X-ray tube 5 at the set SID value d1.

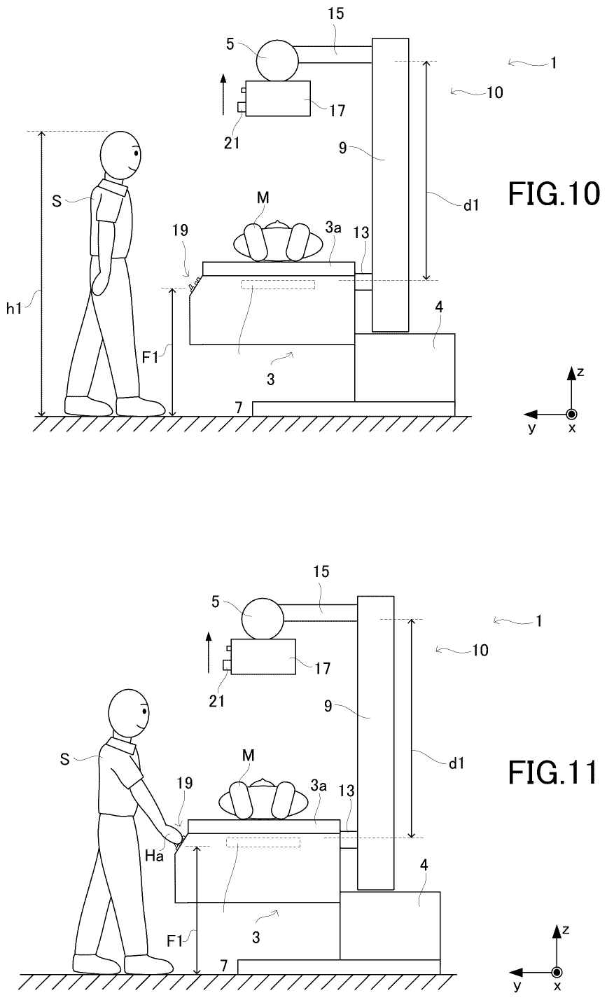

The imaging system drive control unit 41 that receives the control signal controls operation of the X-ray tube driving unit 25, whereby the X-ray tube 5 moves upward. Consequently, as shown in FIG. 10, the distance between the X-ray tube 5 and the X-ray detector 7 is the set SID value d1 while the level of the operating unit 19 is adjusted to the optimum value F1. That is, in the X-ray imaging apparatus 1, the level of the operating unit 19 is adjusted to the optimum value F1, and the distance of the imaging system is automatically adjusted to the set SID value d1.

### Step S6 (Start Procedure of Subject)

The level of the operating unit 19 is adjusted to the optimum value F1, whereby the process in the step S6 starts. That is, as shown in FIG. 11, the operator S touches his/her hand Ha to the operating unit 19 whose level is adjusted to the optimum value F1. The operator S then performs the procedure on the subject M while operating the operating unit 19 with his/her hand Ha. The imaging system or the table 3 is displaced according to input operation of the operator S accepted by the operating unit 19. In other words, a position of an X-ray irradiation field is changed by operating the operating unit 19 by the operator S.

The operator S operates the operating unit 19 to adjust the X-ray irradiation field to an appropriate position. After confirming the position of the X-ray irradiation field, the operator S signals to the technologist C. The technologist C operates the input device 11 according to the signal from the operator S to perform radiography while the X-ray tube 5 irradiates the subject M with X-rays. The operator S performs endoscopy on the subject M while checking the X-ray image of the subject M, generated by the image processing unit 37, with the display unit 31 and the like.

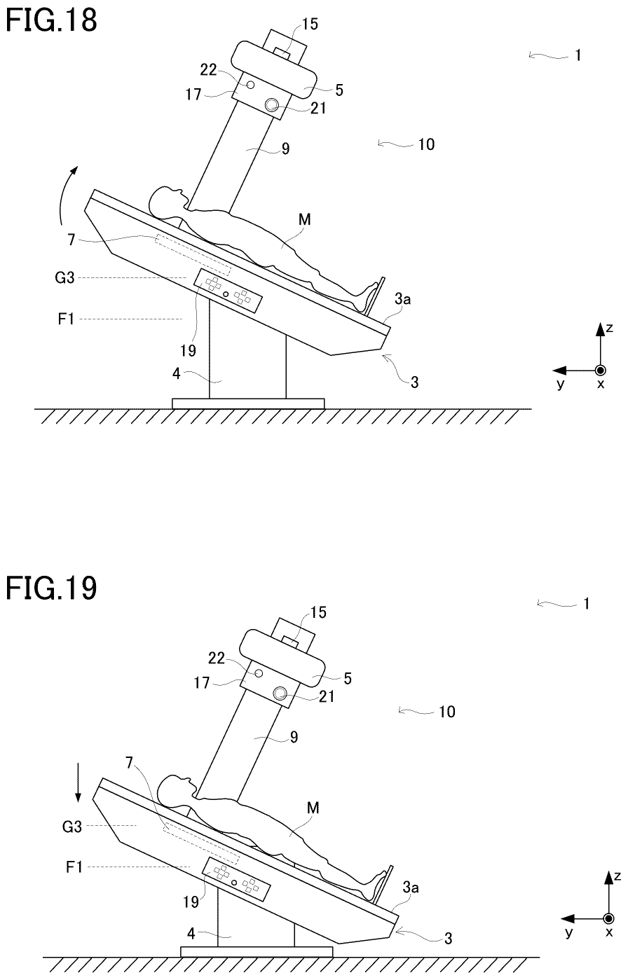

As shown by a numeral T1 in the flowchart of FIG. 4, the process is branched depending on whether or not the level of the operating unit 19 is changed while the procedure is in progress. As an example, while the procedure is in progress, operation to change an angle of the table 3 may be performed occasionally, as shown in FIG. 18. By changing the angle of the table 3, the operating unit 19 arranged on the table 3 is also moved. As a result, the level of the table 3 is changed from the optimum value F1 to a level G3. The operator S performing the procedure on the subject M on the front side of the table 3 is omitted in FIG. 18 for convenience of explanation.

If the level of the operating unit 19 is changed such as by the operation to change the angle of the table 3, a process concerning the step S5 is performed. That is, the level control unit 45 controls the level of the table 3 again when a level sensor, not shown, detects that the level of the operating unit 19 has been changed from the optimum value F1. Under control of the level control unit 45, the level of the operating unit 19 is automatically adjusted from the level G3 to the optimum value F1, as shown in FIG. 19. In this manner, the level control unit 45 always keeps the level of the operating unit 19 at the optimum value F1. Consequently, even when operation to displace the table 3 or the imaging system is performed, the operating unit 19 is automatically adjusted to a position suitable for the operation by the operator S. If the level of the operating unit 19 is not changed, the operator S continues the procedure on the subject M.

When the procedure on the subject M is completed, the operator S moves away from the position on the front side of the table 3. That is, the operator S moves out of the imaging area of the optical camera 21. The technologist C confirms that the operator S has completed the procedure, and operates the input device 11 to move the table 3 downward. That is, the technologist C moves the table 3 downward so that the level of the operating unit 19 moves downward from the optimum value F1 to the lower level G1. After moving the table 3 downward, the operator S lowers the subject M from the placement surface 3a of the table 3. The operator S then leaves the radiography room R1 with the subject M, whereby a series of operations is completed.

### Second Embodiment

The following describes an X-ray imaging apparatus 1A according to a second embodiment of the present invention. Here, same reference numerals are to be merely applied to identify same elements as in the X-ray imaging apparatus 1 described in the first embodiment, and different elements are to be described in detail.

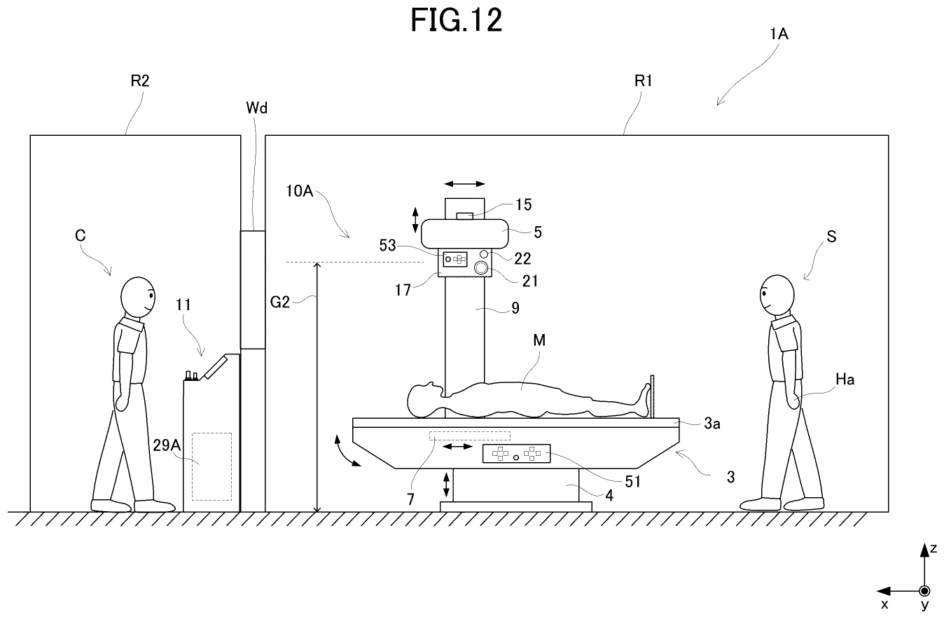

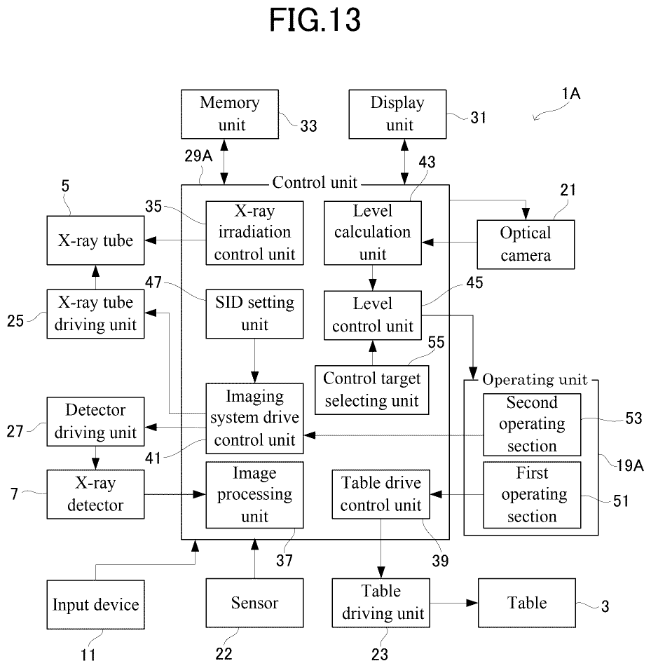

As shown in FIGS. 12 and 13, the X-ray imaging apparatus 1A according to the second embodiment includes an apparatus body 10A, an input device 11, and a control unit 29A. The apparatus body 10A includes an operating unit 19A. The operating unit 19A includes a first operating section 51 and a second operating section 53. That is, the apparatus body 10 of the first embodiment includes the operating unit operated by the operator S at one site, while the apparatus body 10A of the second embodiment has the operating sections at a plurality of sites.

The first operating section 51 is configured to accept input operation of the operator S concerning driving of the table 3. That is, the information received by the first operating section 51 is transmitted to the table drive control unit 39. The first operating section 51 is located on the front face of the table 3, which is similar to the operating unit 19 of the first embodiment. The table 3 has an X-ray detector 7 embedded therein. That is, the first operating section 51 is located at a position whose level changes together with a level of the X-ray detector 7 in the imaging system. In other words, the level of the first operating section 51 changes together with the level of the X-ray detector 7.

The second operating section 53 is configured to accept input operation of the operator S concerning driving of the imaging system. That is, information received by the second operating section 53 is transmitted to the imaging system drive control unit 41. The second operating section 53 is located on a front face of a collimator 17. The collimator 17 is located below the X-ray tube 5. That is, the second operating section 53 is located at a position whose level changes together with a level of the X-ray tube 5 in the imaging system. In other words, the level of the second operating section 53 changes together with the level of the X-ray tube 5.

The control unit 29A according to the second embodiment further includes a control target selecting unit 55. The control target selecting unit 55 selects the first operating section 51 or the second operating section 53 as a target to be controlled by the level control unit 45 according to the type of imaging of the subject M.

The operating section as the target of input operation by the operator S differs between the first operating section 51 arranged on the table 3 and the second operating section 53 arranged on the collimator 17 depending on the type of imaging. When endoscopy with radiography is performed on the subject M as an example, it is easier for the operator S to proceed with the procedure if the operator S adjusts the X-ray irradiation field using the first operating section 51 arranged on the table 3. On the other hand, when a puncture with radiography is performed on the subject M, it is easier for the operator S to proceed with the procedure if the operator S adjusts the X-ray irradiation field using the second operating section 53 arranged on the collimator 17.

Then, as an example, when radiography is performed together with endoscopy on the subject M, the control target selecting unit 55 selects the first operating section 51 as a target to be controlled by the level control unit 45. In this case, the level control unit 45 controls the level of the first operating section 51 according to a physique of the operator S. That is, the level control unit 45 controls up-down movement of the table 3 so that the level of the first operating section 51 is suitable for the operation by the operator S.

On the other hand, when radiography is performed together with the puncture on the subject M, the control target selecting unit 55 selects the second operating section 53 as a target to be controlled by the level control unit 45. In this case, the level control unit 45 controls the level of the second operating section 53 according to a physique of the operator S. That is, the level control unit 45 controls up-down movement of the X-ray tube 5 so that the level of the second operating section 53 is suitable for the operation by the operator S.

In addition, the operating section as the target to be controlled by the level control unit 45 is predetermined from the first operating section 51 and the second operating section 53 depending on the type of imaging. Information of the operating section to be controlled by the level control unit 45, which is tied to each type of imaging, is stored in the memory unit 33. When information identifying the type of imaging for the subject M is inputted with the input device 11, the control target selecting unit 55 reads the information on the operating sections stored in the memory unit 33 and selects the operating section to be controlled by the level control unit 45 according to the type of imaging.

### <Description of Operation>

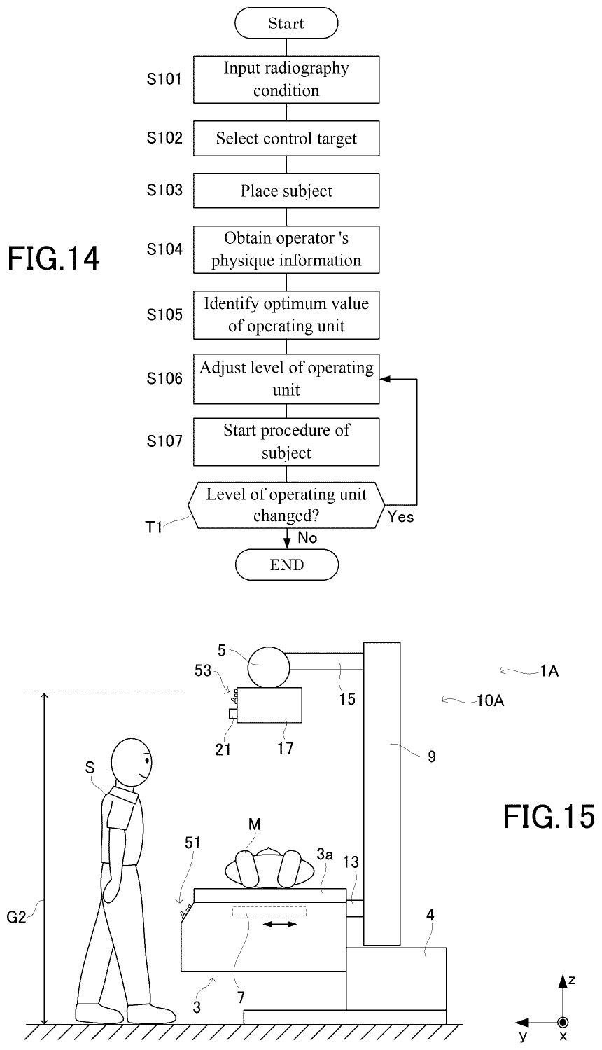

Next, description will be made of operation of the X-ray imaging apparatus 1A according to the second embodiment. FIG. 14 is a flow chart illustrating a summary of operation of a fluoroscopic X-ray apparatus 1A according to the second embodiment. The operation of the X-ray imaging apparatus 1A in the second embodiment differs from that of the X-ray imaging apparatus 1 in the first embodiment in that a process concerning a step S102 is further performed.

As an example of a procedure in the second embodiment, description will be made of a case where endoscopy is performed to the subject M while an X-ray image is taken. That is, in the second embodiment, a level of the second operating section 53 located on the collimator 17 is automatically adjusted according to the physique of the operator S. As shown in FIG. 12, the level of the second operating section 53 in an initial state is assumed to be a level indicated by a numeral G2.

### Step S101 (Input Radiography Condition)

When the X-ray imaging apparatus 1A starts operation, the technologist C firstly inputs a radiography condition. A process of the step S101 is common to the process of the step S1 in the first embodiment. That is, when the step S101 starts, the technologist C enters the control room R2, and the technologist C operates the input device 11 to input an imaging condition of the subject M, for example, values of the SID and a tube voltage. The SID setting unit 47 sets the value of the SID, inputted by the technologist C, as a set SID value d1. The imaging system drive control unit 41 controls the level of the X-ray tube 5 in accordance with the set SID value d1.

Moreover, the technologist C operates the input device 11 to move the table 3 downward in advance before the subject M enters the radiography room R1 (see FIG. 5). That is, the first operating section 51 is lowered to the level G1.

### Step S102 (Select Control Target)

After the imaging condition is inputted, operation to select a control target of the level control unit 45 is performed. When the step S102 starts, the technologist C further operates the input device 11 to input a type of imaging to the subject M. In other words, the technologist C inputs information to perform puncture with radiography.

The information on the type of imaging that the technologist C inputs is transmitted to the control target selecting unit 55. The control target selecting unit 55 reads the information stored in the memory unit 33, and identifies an operating unit 19A corresponding to the type of imaging. When puncture is performed, information that the second operating section 53 is more suitable for operation by the operator S than the first operating section 51 is stored in advance in the memory unit 33. Accordingly, the control target selecting unit 55 selects the second operating section 53 as a target to be controlled by the level control unit 45 when information to perform the puncture is inputted as an imaging type.

The information that the target to be selected by the control target selecting unit 55 is the second operating section 53 is transmitted to the level calculation unit 43 and the level control unit 45. The level calculation unit 43 calculates an optimum value F2 for the second operating section 53 selected by the control target selecting unit 55. Here, the optimum value F2 corresponds to the level of the second operating section 53 that is suitable for input operation by the operator S. The level control unit 45 determines the second operating section 53 of the operating unit 19A selected by the control target selecting unit 55 as a target to be controlled. That is, the level control unit 45 controls the level of the second operating section 53 to the optimum value F2 in the step S106 described below according to selection by the control target selecting unit 55.

### Step S103 (Place Subject)

When the target to be controlled by the level control unit 45 is selected, a process of the step S103 starts. The process of the step S103 is common to the process of the step S2 in the first embodiment. That is, when the step S103 starts, the operator S enters the radiography room R1 with the subject M. Then, the operator S places the subject M on the placement surface 3a of the table 3. In the step S101, the table 3 is moved downward to a level such that the first operating section 51 is at the level G1. Accordingly, the operator S can perform the task of placing the subject M on the table 3 more easily.

In an initial state, the collimator 17 is moved to a position where the second operating section 53 is at the level G2. Since the level G2 is higher than the body height of the operator S, the hand Ha of the operator S in the standing posture does not reach the second operating section 53. When the operator S tries to operate the second operating section 53 while the second operating section 53 is at the level G2, the operator S needs to stretch himself/herself or stretch his/her arm largely, for example. Such a posture is burdensome for the operator S.

Here in the X-ray imaging apparatus 1 according to the second embodiment, the level of the second operating section 53 is automatically controlled to an appropriate level for the operator S by the process of a step S104 to a step S106.

### Step S104 (Obtain Physique Information on Operator)

When the subject M is placed on the table 3, a process of the step S104 starts. The process of the step S104 is common to the process of the step S3 in the first embodiment. That is, as shown in FIG. 15, the operator S moves to a front side of the table 3 to perform a procedure on the subject M. The operator S moves to the front side of the table 3 and approaches the subject M, thereby moving into an imaging area of the optical camera 21. For convenience of explanation, the sensor 22 is omitted in FIG. 15.

When the operator S moves to the front side of the table 3, the operator S is positioned within a detection area of the sensor 22. The sensor 22 detects that the operator S has moved to an imaging area of the optical camera 21. Information detected by the sensor 22 is transmitted from the sensor 22 to the control unit 29. The control unit 29 receives information that the operator S is present within the imaging area of the optical camera 21, thereby controlling the optical camera 21. The optical camera 21 receives a control signal from the control unit 29, and captures an optical image of the operator S. By capturing the optical image of the operator S, information on the physique of the operator S is obtained.

### Step S105 (Identify Optimum Value of Operating Section)

When the optical camera 21 captures the optical image of the operator S, a process of the step S105 starts. In the step S105, an optimum value for a level of the operating section of the operating unit 19A selected by the control target selecting unit 55 is identified. If the type of imaging is radiography performed with the puncture, the second operating section 53 is selected by the control target selecting unit 55. Accordingly, in the step S105, an optimum value F2 of the second operating section 53 is identified.

When the step S105 starts, data of the optical image of the operator S is transmitted from the optical camera 21 to the level calculation unit 43. The level calculation unit 43 analyzes the optical image of the operator S, and calculates the optimum value F2 of the second operating section 53. In the second embodiment, by analyzing the optical image of the operator S, the level calculation unit 43 identifies a body height h1 of the operator S, which is similar in the first embodiment.

The larger the physique of the operator S becomes, the higher the level of the second operating section 53 corresponding to the optimum value F2 is. Accordingly, the level calculation unit 43 calculates the optimum value F2 for the level of the second operating section 53 by multiplying a predetermined coefficient a2 by the value h1 of the body height of the operator S. By using the optical image of the operator S in such a manner, the optimum value F2 is identified for the level of the second operating section 53.

### Step S106 (Adjust Level of Operating Unit)

When the optimum value F2 of the second operating section 53 is identified by the level calculation unit 43, a process of the step S106 starts. That is, information on the optimum value F2 for the level of the second operating section 53 is transmitted from the level calculation unit 43 to the level control unit 45.

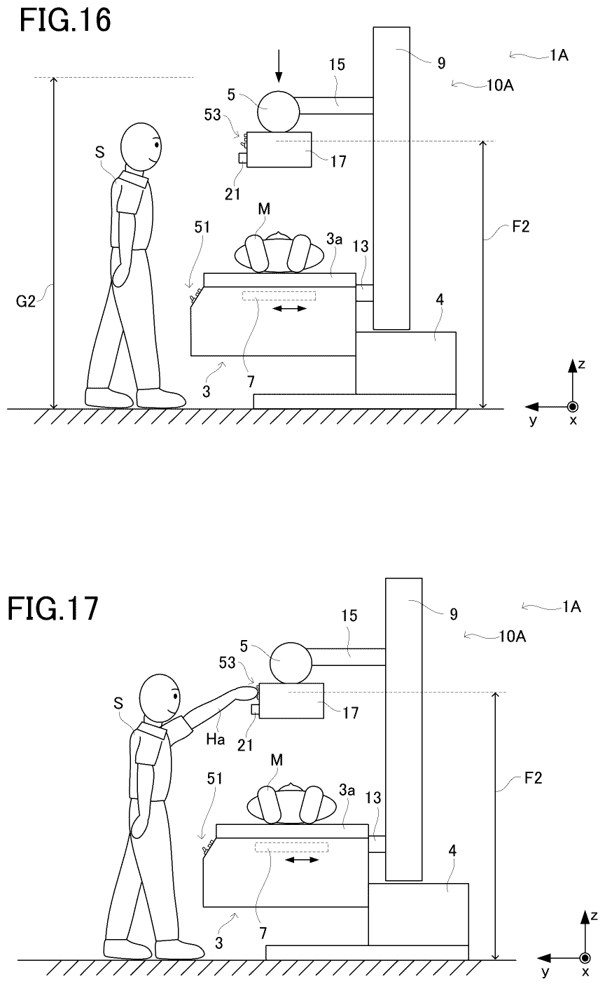

The level control unit 45 controls operation of the various components of the apparatus body 10 so that the level of the second operating section 53 becomes the optimum value F2. In the second embodiment, the second operating section 53 is located on the collimator 17 connected to a lower part of the X-ray tube 5. Accordingly, the level control unit 45 transmits a control signal to the imaging system drive control unit 41. The imaging system drive control unit 41 controls the X-ray tube driving unit 25 to move the collimator 17 downward with the X-ray tube 5. As a result, as shown in FIG. 16, the second operating section 53 moves upward with the X-ray tube 5, and the level of the second operating section 53 changes from the level G2 to the optimum value F2.

By performing the step S104 to the step S106 in such a manner, the level of the second operating section 53 is automatically adjusted to the optimum value F2 when the operator S moves into the imaging area of the optical camera 21. In other words, the level of the second operating section 53 is automatically adjusted to the optimum value F2 while the operator S keeps his/her standing posture. Thus, the level of the second operating section 53 can be adjusted to a position suitable for a physique of the operator S without troublesome movement such as stretching the operator S and stretching the arm of the operator S to a higher position.

Here, as the X-ray tube 5 is moved downward with the second operating section 53, the value of the SID becomes smaller. Accordingly, the SID setting unit 47 transmits a control signal to the table drive control unit 39 to keep the distance between the X-ray detector 7 and the X-ray tube 5 at the set SID value d1.

The imaging system drive control unit 41 that receives the control signal controls operation of the table driving unit 23, whereby the table 3 moves downward. Consequently, the distance between the X-ray tube 5 and the X-ray detector 7 is the set SID value d1 while the level of the second operating section 53 is adjusted to the optimum value F2. That is, in the X-ray imaging apparatus 1A, the level of the second operating section 53 is adjusted to the optimum value F2 and the distance between the elements constituting the imaging system is automatically adjusted to the set SID value d1.

### Step S107 (Start Procedure of Subject)

The level of the second operating section 53 is adjusted to the optimum value F2, whereby a process in a step S107 starts. That is, as shown in FIG. 17, the operator S touches his/her hand Ha to the second operating section 53 whose level is adjusted to the optimum value F2. The operator S then performs the procedure on the subject M while operating the second operating section 53 with his/her hand Ha. A position and an angle of the imaging system are changed according to input operation of the operator S received by the second operating section 53. In other words, a position of an X-ray irradiation field is changed by operating the second operating section 53 by the operator S.

The operator S operates the second operating section 53 to adjust the X-ray irradiation field to an appropriate position. After confirming the position of the X-ray irradiation field, the operator S signals to the technologist C. The technologist C operates the input device 11 according to the signal from the operator S to perform radiography while the X-ray tube 5 irradiates the subject M with X-rays. The operator S performs a puncture on the subject M while checking the X-ray image of the subject M, generated by the image processing unit 37, with the display unit 31 and the like.

As shown by a numeral T1 in the flowchart of FIG. 14, if the level of the operating unit 19 is changed from the optimum value F2 to the level G3 while the procedure is in progress, the process concerning the step S5 is performed. That is, the level control unit 45 controls the level of the table 3 again when a level sensor, not shown, detects that the level of the operating unit 19 has been changed from the optimum value F1. Under control of the level control unit 45, the level of the operating unit 19 is automatically adjusted from the level G3 to the optimum value F2. In this manner, the level control unit 45 always keeps the level of the operating unit 19 at the optimum value F2. Consequently, even when an operation to displace the table 3 or the imaging system is performed, the operating unit 19 can be adjusted automatically to a position suitable for operation by the operator S. If the level of the operating unit 19 is not changed, the operator S continues the procedure on the subject M.

When the procedure on the subject M is completed, the operator S moves away from the position on the front side of the table 3. That is, the operator S moves out of the imaging area of the optical camera 21. The technologist C confirms that the operator S has completed the procedure, and operates the input device 11 to move the table 3 downward. After moving the table 3 downward, the operator S lowers the subject M from the placement surface 3a of the table 3. The operator S then leaves the radiography room R1 with the subject M, whereby a series of operations is completed.

### <Effect of Construction in Embodiments>

(First paragraph) The X-ray imaging apparatus 1 according to the present embodiments includes the X-ray tube 5 that irradiates the subject M with X-rays, the X-ray detector 7 that detects X-rays transmitted through the subject M, the operating unit 19 that is located at a position where a level thereof changes together with a level of at least either the X-ray tube 5 or the X-ray detector 7 constituting an imaging system and receives input operation by the operator S, the optical camera 21 that acquires information on a physique of the operator S, and the level control unit 45 that controls a level of the operating unit 19 to be the optimum value F1 suitable for the input operation by the operator S in accordance with the physique information.

According to the X-ray imaging apparatus described in a first paragraph, the imaging system consisting of the X-ray tube 5 and the X-ray detector 7, the operating unit 19 that receives input operation by the operator S, the optical camera 21, and the level control unit 45 are included. The level of operating unit 19 changes together with the level of at least one constituting the imaging system.

The optical camera 21 acquires information on the physique of the operator S. The level control unit 45 controls the level of the operating unit 19 to the optimum value F1 based on the information on the physique of the operator S obtained by the optical camera 21. The optimum value F1 corresponds to the level of the operating unit 19 that is suitable for input operation by the operator S.

That is, when the optical camera 21 acquires the information on the physique of the operator, the level of the operating unit 19 is controlled automatically to be suitable for input operation by the operator S. Accordingly, even if the operating unit 19 is moved to a position where it is difficult for the operator S to operate due to placing the subject M on the table of the X-ray imaging apparatus 1, the operating unit 19 is automatically moved to the suitable level F1 by the optical camera 21 and the level control unit 45. This can avoid a situation in which a burden on the operator S increases due to movement of the operating unit 19 to a position where it is difficult for the operator to operate. This can also prevent extended time required for the procedure to the subject M.

(Second paragraph) In the X-ray imaging apparatus described in the first paragraph, provided are the SID setting unit 47 that sets the value of the SID, which is the distance between the X-ray tube 5 and the X-ray detector 7, and the imaging system drive control unit 41 that controls the level of the X-ray tube 5 or the X-ray detector 7 so as to keep the set SID value d1 set in advance by changing the level of the operating unit 19 by the level control unit 45.

According to the X-ray imaging apparatus described in the second paragraph, the SID setting unit 47 and the imaging system drive control unit 41 are provided. The SID setting unit 47 sets the value of the SID, which is the distance between the X-ray tube 5 and the X-ray detector 7, as the set SID value d1. When the level control unit 45 changes the level of the operating unit 19, the imaging system drive control unit 41 controls the level of the X-ray tube 5 or the X-ray detector 7 to maintain the set SID value d1 in advance.

With this configuration, even if the level of the operating unit 19 is changed with one constituting the imaging system, the level of the other constituting the imaging system is automatically adjusted by the imaging system drive control unit 41. As a result, the distance between the elements of the imaging system can be kept at the set SID value d1 while the level of the operating unit 19 is adjusted to the optimum value F1. Consequently, it is possible to avoid deviation of the SID value from a proper value and degradation in quality of the X-ray image due to such a configuration that the level of the operating unit 19 is adjusted to the optimum value F1.

(Third paragraph) In the X-ray imaging apparatus described in the first paragraph, the operating unit 19A includes the first operating section 51 arranged at a position where the level thereof is changed together with the level of one constituting the imaging system, and the second operating section 53 arranged at a position where the level thereof is changed together with the level of the other constituting the imaging system, and also includes the control target selecting unit 55 that selects a target to be controlled by the level control unit 45 from the first operating section 51 and the second operating section 53 according to the type of imaging of the subject M.

According to the X-ray imaging apparatus described in the third paragraph, the control target selecting unit 55 is provided. Moreover, the operating unit 19A includes the first operating section 51 and the second operating section 53. The control target selecting unit 55 selects a target of control by the level control unit 45 from the first operating section 51 or the second operating section 53 according to the type of imaging of the subject M.

The first operating section 51 is located at a position whose level changes together with the level of one constituting the imaging system. The second operating section 53 is located at a position whose level changes together with the level of the other constituting the imaging system. The first operating section 51 of the operating unit 19A may be suitable for operation by the operator S depending on the type of imaging. On the other hand, the second operating section 53 of the operating unit 19A may be suitable for operation by the operator S if the type of imaging is different.

In the X-ray imaging apparatus 1A, the control target selecting unit 55 automatically selects a target to be controlled by the level control unit 45 by identifying the type of imaging even if the operating unit 19A includes multiple operating sections. This avoids a situation where the level of the operating section in the operating unit 19A that is not suitable for operation by the operator S is automatically controlled. Moreover, troublesome operation of manually selecting an operating section as the target to be controlled for each type of imaging by the level control unit 45 can be omitted.

(Fourth paragraph) In the X-ray imaging apparatus described in the first paragraph, the physique information acquiring unit includes the optical camera 21 that captures an image of the operator S, and the level calculation unit 43 that calculates the optimum value F1 for the level suitable for input operation by the operator S based on the image of the operator S captured by the optical camera 21.

According to the X-ray imaging apparatus described in the fourth paragraph, information on the physique of the operator S is obtained by the optical camera 21 and the level calculation unit 43. The optical camera 21 captures an image of the operator S. The level calculation unit 43 uses the image of the operator S to calculate the optimum value F1. More accurate information on the physique of the operator S can be obtained, such as the body height h1 of the operator S or the level of the operator's shoulders by capturing the image of the operator S. The level calculation unit 43 can calculate the optimum value F1 according to a physique of an operator S by calculating the optimum value F1 with use of an image of the operator S. Consequently, even if another operator S performs a procedure on a subject M, it is possible to identify the optimum value F1 of the operating unit 19 according to the physique of the operator S who performs the procedure more precisely.

### Other Embodiments

The embodiments disclosed here are all illustrative in every aspect, but not restrictive. The scope of the invention includes claims and all modifications within the meaning and range equivalent to the claims. As one example, the invention may be modified as follows.

(1) In the X-ray imaging apparatus described above, the optical camera 21 that captures an optical image of the operator S is exemplarily described as the configuration for acquiring information on the physique of the operator S, but this is not limitative. As an example, an infrared camera that captures an infrared image of the operator S may be used. Various sensors, such as an infrared sensor, may also be used to detect the physique of the operator S by scanning the operator S.

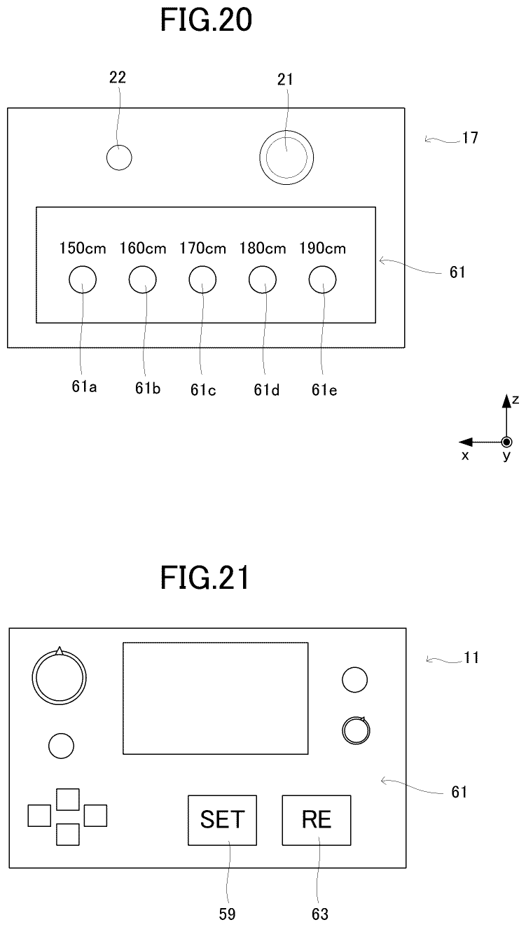

(2) In the X-ray imaging apparatus described above, the configuration for obtaining information on the physique of the operator S may be such that the operator S or the technologist C inputs information on the physique of the operator S. Examples of the configuration for inputting information on the physique of the operator S include a physique information input unit 61 as shown in FIG. 20. The physique information input unit 61 is located on the collimator 17, the strut 9, or the input device 11, as an example.

The physique information input unit 61 has a plurality of input buttons 61a to 61e. The input buttons 61a to 61e are provided for each predetermined step value of the body height. The input button 61a is operated when information that the body height of the operator S is 150 cm or less is to be inputted. The input button 61b is operated when information that the body height of the operator S is 160 cm is to be inputted. The input button 61c is operated when information that the body height of the operator S is 170 cm is to be inputted. The input button 61d is operated when information that the body height of the operator S is 180 cm is to be inputted. The input button 61e is operated when information that the body height of the operator S is 190 cm or more is to be inputted.

As an example, if the body height of the operator S is 150 cm or less, the operator S or the technologist C presses the input button 61a. As an example, if the body height of the operator S is 180 cm, the operator S or the technologist C presses the input button 61d. The physique information input unit 61 receives information entered by the operator S or the technologist C, and transmits the received body height information to the control unit 29. Thus, by operating the input buttons 61a to 61e corresponding to the body height of the operator S in such a manner, information on the physique of the operator S is obtained. The level calculation unit 43 calculates the optimum value F1 or the optimum value F2 based on the obtained information on the physique of the operator S.

Examples of a timing for operating input buttons 61a to 61e include the step S1 or the step S2. The physique information input unit 61 is not limited to a configuration using the input buttons 61a to 61e as button-type switches, but may also be configured to input values of the body height of the operator S by dial input or keyboard input.

(3) In the X-ray imaging apparatus described above, the optical camera 21 that captures an optical image of the operator S is not limited to the configuration in which it is located in the collimator 17. Other examples of positions where the optical camera 21 is located include the strut 9 or the ceiling of a radiography room R1.

(4) In the X-ray imaging apparatus described above, the level control unit 45 controls the level of the operating unit 19 to the optimum value F1, which is triggered by receiving information on the optimum value F1 from the level calculation unit 43 as an example, but is not limited to this. As an example, the X-ray imaging apparatus 1 includes a level adjustment instruction switch 59, not shown, and the level control unit 45 may control the level of the operating unit 19 to the optimum value F1, which is triggered by operating the level adjustment instruction switch 59 after the optimum value F1 is calculated.

The level adjustment instruction switch 59 is arranged on the input device 11 or the apparatus body 10 as one example. FIG. 21 shows one example of the input device 11 where the level adjustment instruction switch 59 is arranged. In one modification with the level adjustment instruction switch 59, the level control unit 45 receives information on the optimum value F1 from the level calculation unit 43, which triggers a state where the level adjustment instruction switch 59 is ready for input operation (activated state). When the level adjustment instruction switch 59 is in the activated state, the operator S or the technologist C is notified by light or a sound that the level adjustment instruction switch 59 has been activated. As an example of a configuration to notify that the level adjustment instruction switch 59 has been activated, a light source located on the level adjustment instruction switch 59 blinks.

When the level adjustment instruction switch 59 is arranged on the input device 11, the level control unit 45 controls the level of the operating unit 19 to the optimum value F1 by the technologist C pressing the level adjustment instruction switch 59 in the activated state. When the level adjustment instruction switch 59 is arranged on the apparatus body 10, the level control unit 45 controls the level of the operating unit 19 to the optimum value F1 when the operator S presses the level adjustment instruction switch 59 in the activated state.

Thus, in the configuration of the modification with the level adjustment instruction switch 59, a timing for controlling the level of the operating unit 19 to the optimum value F1 can be adjusted appropriately in this way. That is, when the optical camera 21 captures the optical image of the operator S and the level calculation unit 43 calculates the optimum value F1, the level adjustment instruction switch 59 is brought into the activated state. The technologist C or the operator S confirms that the level adjustment instruction switch 59 has been activated, and presses the level adjustment instruction switch 59 at a desired timing. The level control unit 45 receives the input operation of pressing the level adjustment instruction switch 59, thereby controlling the level of the operating unit 19 to the optimum value F1. A timing for controlling the level of the operating unit 19 to the optimum value F1 is set not to a timing that the level control unit 45 receives the information on the optimum value F1 from the level calculation unit 43 but to a timing when the level adjustment instruction switch 59 is pressed, leading to prevention of change in the level of the operating unit 19 at a timing that is inconvenient for the technologist C or the operator S.

Moreover, in the configuration of the modification with the level adjustment instruction switch 59, when the level of the operating unit 19 is changed from the optimum value F1 to the level G3 during the progress of the procedure concerning the step S6, the technologist C or the operator S presses the level adjustment instruction switch 59. The level control unit 45 receives the input operation of the level adjustment instruction switch 59, whereby the level of the operating unit 19 is changed to the optimum value F1. That is, there is no need to operate the operating unit 19 depending on the progress of the procedure. Moreover, the condition where the operating unit 19 is at the level G3 may sometimes be more suitable for the progress of the procedure than the condition where the operating unit 19 is at the level of the optimum value F1. If there is no need to adjust the level of the operating unit 19 to the optimum value F1, the operator S continues the procedure without operating the level adjustment instruction switch 59. Since the level adjustment instruction switch 59 is provided in this way, the operator S can decide whether or not and a timing when the level of the operating unit 19 is changed to the optimum value F1, which is the level suitable for operating the operating unit 19.

(5) In the modification with the level adjustment instruction switch 59, a level return instruction switch 63 may also be provided. The level return instruction switch inputs an instruction to return the level of the operating unit 19 to a pre-adjusted level when the level adjustment instruction switch 59 adjusts the level of the operating unit 19 to the optimum value F1. The level return instruction switch 63 is arranged on the input device 11 or the apparatus body 10 as one example, which is similar to the level adjustment instruction switch 59. FIG. 21 shows one example of the input device 11 where the level adjustment instruction switch 59 and the level return instruction switch 63 are arranged.

In the modification in which the level adjustment instruction switch 59 and the level return instruction switch 63 are provided, when the level of the operating unit 19 is changed from the optimum value F1 to the level G3 during the progress of the procedure concerning the step S6, the technologist C or the operator S presses the level adjustment instruction switch 59. The level control unit 45 receives the input operation of the level adjustment instruction switch 59, and adjusts the level of the operating unit 19 from the level G3 to the optimum value F1. At this time, the operation of pressing down the level adjustment instruction switch 59 triggers the memory unit 33 to store the level of the operating unit 19 at the time when the level adjustment instruction switch 59 is pressed. That is, the memory unit 33 stores a value of the level G3 as the level of the operating unit 19 before adjustment.

When the level of the operating unit 19 is changed to the optimum value F1, the operator S operates the operating unit 19, which has been changed to a level suitable for input operation, to adjust an X-ray irradiation field and the like. After the input operation of the operating unit 19 is completed, the operator S returns the level of the operating unit 19 from the optimum value F1 to the level G3 by pressing the level return instruction switch 63. That is, the input operation of the level return instruction switch 63 is accepted by the level control unit 45.

When the level control unit 45 accepts the input operation of the level return instruction switch 63, information on the level of the operating unit 19 before adjustment is read out from the memory unit 33. The level control unit 45 then controls the table drive control unit 39 or the imaging system drive control unit 41 in accordance with the information read out, and adjusts the level of the operating unit 19 so that the level of the operating unit 19 becomes the level of the operating unit 19 at the time when the level adjustment instruction switch 59 is pressed. As a result, the level of the operating unit 19 returns from the optimum value F1 to the level G3, which is triggered by the operation of pressing the level return instruction switch 63.

When the level of the operating unit 19 returns to the level G3, the operator S resumes the procedure on the subject M in the apparatus body 10 whose level is returned to a level suitable for the procedure. The level adjustment instruction switch 59 and the level return instruction switch 63 are provided in this manner, whereby the level of the operating unit 19 can be changed appropriately between a level suitable for input operation of the operating unit 19 and a level suitable for performing the procedure on the subject M in the process of the procedure concerning the step S6. Here, the configurations according to the modifications in the above (4) and (5) may be applied to either the X-ray imaging apparatus 1 according to the first embodiment or the X-ray imaging apparatus 1A according to the second embodiment.

### REFERENCE SIGNS LIST

1 . . . X-ray imaging apparatus
3 . . . table
5 . . . X-ray tube
7 . . . X-ray detector
10 . . . apparatus body
11 . . . input unit
17 . . . collimator
19 . . . operating unit
21 . . . optical camera
22 . . . sensor
23 . . . table driving unit
25 . . . X-ray tube driving unit
27 . . . detector driving unit
29 . . . control unit
37 . . . image processing unit
39 . . . table drive control unit
41 . . . imaging system drive control unit
43 . . . level calculation unit
45 . . . level control unit
47 . . . SID setting unit
51 . . . first operating section
53 . . . second operating section
55 . . . control target selecting unit
M . . . subject
S . . . operator
C . . . technologist

What is claimed is:

1. An X-ray imaging apparatus, comprising: an X-ray tube that irradiates a subject with X-rays;
an X-ray detector that detects X-rays transmitted through the subject;
an operating unit that is located at a position where a level thereof changes together with a level of at least either the X-ray tube or the X-ray detector constituting an imaging system and accepts input operation by an operator;
a physique information acquiring unit that acquires information on a physique of the operator; and
a level control unit that controls the level of the operating unit to be suitable for the input operation by the operator in accordance with the information on the physique.

2. The X-ray imaging apparatus according to claim 1, further comprising:
an SID setting unit that sets a value of an SID, which is a distance between the X-ray tube and the X-ray detector; and
an imaging system control unit that controls the level of the X-ray tube or the X-ray detector so as to keep the value of the SID set in advance by changing the level of the operating unit by the level control unit.

3. The X-ray imaging apparatus according to claim 1, wherein
the operating unit includes a first operating section that is arranged at a position where a level thereof is changed together with a level of one constituting the imaging system, and a second operating section that is arranged at a position where a level thereof is changed together with a level of the other constituting the imaging system, and
also includes a control target selecting unit that selects a target to be controlled by the level control unit from the first operating section and the second operating section according to a type of imaging of the subject.

4. The X-ray imaging apparatus according to claim 1, wherein
the physique information acquiring unit includes an imaging unit that captures an image of the operator, and
includes a level calculation unit that calculates a level suitable for input operation by the operator based on the image of the operator captured by the imaging unit.
